(19) United States
(12) Patent Application Publication
(10) Pub. No.: US 2002/0082177 A1
(43) Pub. Date: Jun. 27, 2002

(54) Method to provide assurance that skin has been washed and subsequently sanitized

(76) Inventor: Irvin Lance Tabaac, New Hope, PA (US)

Correspondence Address: Irvin L. Tabaac, 49 Old York Road, New Hope, PA, 18938, US

(21) Appl. No.: 09/747,264

(22) Filed: Dec. 26, 2000

(51) Int. Cl. A61K 7/50

(52) U.S. Cl. 510/130; 510/137

(57) ABSTRACT

A method, staining compositions, and stain removal compositions are disclosed which provide assurance of the washing and subsequent sanitization of skin, particularly skin located on human hands. The hands are washed with a first composition containing at least a soap, detergent, or other skin cleaning agent and an indicator that has the ability to stain certain tissue. The washed yet stained hands are rinsed and dried and then are subjected to a light source with certain wavelengths causing the illumination of the stain on the skin. The hands are then subjected to the application of a second composition that removes the stain and sanitizes the skin. The hands are subjected again to the light source. The presence of the stain on the skin after the first composition is applied and the absence after the second composition is applied indicates satisfactory cleansing and sanitization.

### BACKGROUND OF THE INVENTION

In the health care industries, the food industries, the drug industries, the cosmetic industries and other industries, there is a genuine need to assure the washing and sanitization of skin, especially skin on the hands, prior to the performance of certain tasks. An example of a specific need for assuring sanitized hands is in the dispensing of medications. Another example of a specific need for assuring sanitized hands is in the preparation of foods. It is not desirous to contaminate foods, drugs, or cosmetics with microbes such as a bacteria, yeasts, molds, prions, or viruses. Books such as the United States Pharmacopeia and National Formulary (USP24/NF19) (the United States Pharmacopeial Convention, Inc., 12601 Twin Brook Parkway, Rockville, Md. 20852) list testing methods and limits for microbials. Additionally, the Code of Federal Regulations: 21CFR211.52, 21CFR211.28 and 21CFR211.56 describe personnel responsibilities, washing facilities, toilet facilities and sanitation requirements to prevent the contamination of drug products during the manufacturing, processing and packaging of drugs.

Methods and systems have been patented for helping to assure the washing of hands such as U.S. Pat. Nos. 5,952,924 and 6,031,461. Still another U.S. Pat. No. 5,951,965, describes the method for visually demonstrating the effectiveness of an anti-bacterial attachment composition. The need for adequate hand cleansing is obvious with the allowance of hand cleansing compositions such as those described in U.S. Pat. Nos. 4,263,284 and 4,812,253. The need for proper toilet use and individual hygiene is still further recognized by the allowance of letter patents such as U.S. Pat. No. 5,870,015.

It is also well known to those trained in the art that staining of skin tissue, cells, and plaque can be accomplished by use of various dyes and techniques. U.S. Pat. Nos. 4,431,628 and 5,460,800 and 5,250,223 teach of such staining techniques.

Proper hand cleaning is essential to good hygienic practices. Likewise, it is of paramount importance in the food, pharmaceutical, cosmetic and health care industries to reduce contamination to foods, pharmaceuticals, cosmetics and health care recipients by proper cleansing of hands. Many devices address this issue by flushing the urinal and toilet automatically after use or by automatically lowering a toilet seat into position. Yet, despite these modern day devices, the assurance of a visual demonstration of effective hand cleaning is heretofore unsatisfied.

### SUMMARY OF THE INVENTION

The present invention relates to the satisfactory cleansing and sanitization of skin effected by the application of a first composition containing a visualizable label for staining cells, the visualization of said cells, the removal of said stain by use of a second composition and finally the visualization of the absence of the stain.

### DETAILED DESCRIPTION OF THE INVENTION

It has now been discovered that dyes, such as those classified as “Natural Coloring Principles” and those classified as “Synthetic Coloring Principles” can stain skin. Natural Coloring Principles are obtained from mineral, plant and animal sources. They include red ferric oxide, yellow ferric oxide, titanium dioxide, carbon black, chlorophyll, anattenes, betacarotene, alizarin, indigo, flavones, riboflavin, rutin, hesperidin, quercetin, saffron, cudbear, red saunders, tyranian purple, cochineal, carmine, caramel and other colorants, dyes or pigments from natural sources. Synthetic Coloring Principles are those which can be synthesized. They include 6,6′-dibromoindigo, mauveine, coal-tar dyes, aniline colors, nitroso-dyes, nitrodyes, azo-dyes, oxazines, thiazines, pyrazolones, xanthenes, indigoids, anthraquinones, acridines, rosanilines, phthaleins, quinolines acid dyes, basic dyes, direct dyes, mordant dyes, chromophore containing dyes, auxochrome containing dyes, FD&C Blue #1, FD&C Blue #2, FD&C Green #2, FD&C Red #3, D&C Red#40, D&C Yellow #5, D&C Yellow #6, D&C Blue #6, D&C Green #6, D&C Orange #5, D&C Orange #10, D&C Orange #17, D&C Red #6, D&C Red #7, D&C Red #21, D&C Red #27, D&C Red #28, D&C Red #30, D&C Red #8, D&C Red #12, D&C Red #19, D&C Red #33, D&C Red #36, D&C Yellow #10, Lake Dyes, D&C Yellow #8, D&C RED #28, D&C Green #8 and other colorants dyes or pigments produced synthetically.

More specifically, it has been discovered that dyes such as those listed above and more specifically D&C Yellow #8, D&C Red #28 and D&C Green #8, when used alone or combined, have the ability to stain nail, nail bed, callus, collagen, scar and certain other skin tissue. Even more specifically, it has been discovered that dyes such as D&C Yellow #8, D&C Red #28 and D&C Green #8 when used in concentrations from about 0.00002% to about 0.25% when incorporated into a soap, detergent, shampoo or other skin cleansing agent, stain nail, nail bed, callus, collagen, scar and other skin tissue. This selective staining indicates that a first composition washing with a skin cleansing agent and stain has transpired; this is verified by subjecting the stained tissue to a light source, the light source being of <20,000 angstrom units. More specifically, a light source of ultra violet, visible or infra red wavelengths is desired. Most specifically, a light source of ultra violet wavelengths is desired.

The stain on the nail, nail bed, callus, collagen, scar and certain other skin tissue is then removed by washing, rinsing, rubbing or scrubbing with a second composition containing an agent which dissolves, neutralizes, or otherwise removes the staining compound. This second composition will typically be of such ingredients that it additionally have anti-bacterial or anti-microbial activity. This second composition shall contain at least one of the following ingredients: an alcohol (e.g. Isopropyl Alcohol 5 to 99%; Ethyl Alcohol 5 to 99%); glycerin 2% to 70%; an acid (e.g. Benzoic acid); an aldehyde (e.g. formaldehyde); a phenol (e.g. methyl p-hydroxybenzoate); a quaternary compound (e.g. chlorhexidine); or any other suitable solvent having the capability to dissolve or remove the dye stain. In addition, the second composition shall have as one of its ingredients a compound or solvent which will sanitize the skin. Examples of such sanitizing compounds include: isopropyl alcohol; ethyl alcohol; benzalkonium chloride; triclosan; triclocarban; cationic detergent; green soap; hexachlorophene; selenium sulfide; sodium lauryl sulfate; or other commonly used antiseptic, anti-bacterial or sanitizing compound. After the skin has been subjected to the second composition along with washing, rinsing, rubbing or scrubbing with such composition, the skin is preferably dried by air or clean towel. The skin is then subjected again to a light source, the light source being of <20,000 angstrom units. More specifically, a light source of ultra violet, visible or infra red wavelength is desired. Most specifically, a light source of ultra violet wavelengths is desired. The absence of stain is then to be observed. The second composition, when applied to the skin after the first composition is applied, solubilizes and removes the initial stain.

Thus, when one views the stain after washing with the first composition and then one views the absence of said stain after application of the second composition, one can instantly conclude that proper hand washing and sanitization has been executed.

Accordingly I claim,

1. A method for producing a stain on nail, callous, collagen, nail bed, and other skin tissue by the application of a soap, detergent, shampoo, or other skin cleansing agent to which the staining dye D&C Yellow #8 in a concentration from 0.00002% to 0.25% has been added and said resulting staining composition not being totally removed by water rinsing or towel drying.

2. The method of claim 1, wherein the stain is then visualized by subjecting the stained skin to a light source emitting light within the ultraviolet wavelength.

3. The method of claim 2, wherein the skin is furthermore subject to a washing, rinsing, rubbing or scrubbing with a second composition, said second composition having the ability to solubilize and effectively cause the removal of the staining compound from the skin, also said second composition having the ability to cause a reduction or removal of microbes from the skin, said second composition containing a minimum of one alcohol in a concentration from 5% to 99%.

4. The method of claim 3, wherein the absence of stain is then ascertained by subjecting the skin to a light source emitting light within the ultraviolet wavelength.

5. A method for producing a stain on nail, callous, collagen, nail bed, and other skin tissue by the application of a soap, detergent, shampoo, or other skin cleansing agent to which the staining dye D&C Red #28 in a concentration from 0.00002% to 0.25% has been added and said resulting staining composition not being totally removed by water rinsing or towel drying.

6. The method of claim 5, wherein the stain is then visualized by subjecting the stained skin to a light source emitting light within the ultraviolet wavelength.

7. The method of claim 6, wherein the skin is furthermore subject to a washing, rinsing, rubbing or scrubbing with a second composition, said second composition having the ability to solubilize and effectively cause the removal of the staining compound from the skin, also said second composition having the ability to cause a reduction or removal of microbes from the skin, said second composition containing a minimum of one alcohol in a concentration from 5% to 99%.

8. The method of claim 7, wherein the absence of stain is then ascertained by subjecting the skin to a light source emitting light within the ultraviolet wavelength.

9. A method for producing a stain on nail, callous, collagen, nail bed, and other skin tissue by the application of a soap, detergent, shampoo, or other skin cleansing agent to which the staining dye D&C Green #8 in a concentration from 0.00002% to 0.25% has been added and said resulting staining composition not being totally removed by water rinsing or towel drying.

10. The method of claim 9, wherein the stain is then visualized by subjecting the stained skin to a light source emitting light within the ultraviolet wavelength.

11. The method of claim 10, wherein the skin is furthermore subject to a washing, rinsing, rubbing or scrubbing with a second composition, said second composition having the ability to solubilize and effectively cause the removal of the staining compound from the skin, also said second composition having the ability to cause a reduction or removal of microbes from the skin, said second composition containing a minimum of one alcohol in a concentration from 5% to 99%.

12. The method of claim 11, wherein the absence of stain is then ascertained by subjecting the skin to a light source emitting light within the ultraviolet wavelength.
